(19) United States
(12) Patent Application Publication
(10) Pub. No.: US 2009/0118686 A1
(43) Pub. Date: May 7, 2009

(54) COLOSTOMY ARRANGEMENTS

(75) Inventor: Michael Albert Playdon, Walsall (GB)

Correspondence Address: SMITH-HILL AND BEDELL, P.C., 16100 NW CORNELL ROAD, SUITE 220, BEAVERTON, OR, 97006, US

(73) Assignee: BMTLIFESTYLE LIMITED

(21) Appl. No.: 11/911,152

(22) PCT Filed: Apr. 12, 2006
(86) PCT No.: PCT/GB06/01301

§ 371 Date: Nov. 29, 2007

(30) Foreign Application Priority Data
Apr. 14, 2005 (GB) 0507501.5
May 24, 2005 (GB) 0510597.8
Dec. 19, 2005 (GB) 0525718.3

(51) Int. Cl. A61F 5/44

(52) U.S. Cl. 604/332

(57) ABSTRACT

A bag arrangement for a colostomy stoma, the bag arrangement comprising a bag having a stoma opening and a vent filter, the arrangement including a hood at least secured over the vent filter, the hood including an adaptor to allow gaseous withdrawal through the vent filter.

The present invention relates to colostomy arrangements and more particularly with regard to evacuation of colostomy arrangements through stoma exits.

In order to treat numerous medical complaints it is necessary to perform colostomy surgical procedures in order to remove a part of the intestine and/or bowel. As a result of such colostomy procedures an individual is left with a section of intestine which is directly evacuated through a stoma exit in the abdomen wall. In such circumstances individuals can continue relatively normal lives and faeces and other waste pass through the stoma into a receptacle bag attached to the stoma.

It will be understood that natural bowel movements are facilitated and helped by gravitational factors. However, as a stoma exit is generally located as indicated above in an abdomen wall, gravity has less effect. In any event, an individual after colostomy surgery must relearn how to control and regulate their bowl movements. Previously, such control has been achieved principally through trial and error. It will be understood that in situations where wastes will be collected in bags it is desirable to achieve regularity with regard to bowel movements. Such regularity with bowel movement is normally advantageous but with respect to individuals after colostomy surgery, the ability to predictably expect to change their bag has clear benefits. It will also be understood that as part of the natural digestion process gases will be created and individuals and colostomy arrangements must be able to cope with such gas effusions.

In accordance with aspects of the present invention there is provided a bag arrangement for a colostomy stoma, the bag arrangement comprising a bag having a stoma opening and a vent filter, the arrangement including a hood at least secured over the vent filter, the hood including an adaptor to allow gaseous withdrawal through the vent filter.

Typically, the hood substantially surrounds the whole arrangement with an aperture through which the stoma opening projects.

Typically, the bag is wet proof. Possibly, the bag is formed from a plastics material or a rubber material.

Typically, the hood is fibrous. Possibly, the hood is formed from a textile or non woven felt sheet.

Possibly, the adaptor is sealable. Possibly, the adaptor is sealable by an adhesive patch extending across the adaptor. Alternatively, the adaptor is sealable by a flap displaceable when gaseous withdrawal is provided. Typically, the adaptor comprises a single hole. Alternatively, the adaptor comprises a plurality of holes or perforations.

Generally, the filter is located directly opposite the stoma opening.

Advantageously, the filter is directly aligned with the adaptor.

Generally, the filter is formed from a fibrous material. Generally, the fibrous material is provided by an air laid paper or non woven fabric or wool or foam.

Possibly, the filter is impregnated with a scenting agent.

Advantageously, the filter is integrally secured to the bag. Potentially, the filter provides a moisture barrier when wet.

Possibly, the filter and/or the adaptor are arranged to regulate rates of gaseous withdrawal in use.

Also in accordance with aspects of the present invention there is provided a method of stimulating bowel movements in a colostomy patient, the method using a funnel associated with a gaseous withdrawal pump, the funnel positioned relative to a stoma and the pump operated to draw gas through this stoma for a desired period of time to remove gas by effusion from accumulated bowel waste in a bowel and such effusion stimulating by co withdrawal movements of the bowel waste itself.

Typically, the desired period of time is sufficient to draw gas for bowel waste de-gasification.

Also in accordance with certain aspects of the present invention there is provided a process for stimulation of bowel movements and control, the process comprising a bag arrangement and a method as described above, the bag positioned with the stoma opening over the stoma and the withdrawal pump secured to the adaptor to draw gas through the filter from the stoma to stimulate movement of the bowel waste towards the bag.

Generally, the bag arrangement is retained attached to the stoma when the withdrawal pump is removed.

Typically, the process includes selective use of the withdrawal pump in order to train bowel movements and control.

Further in accordance with the present invention there is provided a bag arrangement for a colostomy stoma, the bag arrangement comprising a bag with a stoma opening and a vent filter for gas, the arrangement having a cover with a concertina pocket to receive gas from the vent filter by expansion and having gas releases elements.

Typically, the gas release elements comprise holes. Typically, the holes are round or oblong to vary aperture widths dependent upon expansion of the concertina pocket. Alternatively, the gas release elements comprise diffusion through the material of the cover.

Typically, the concertina pocket comprises a top and foldable walls. Generally, the gas release elements are located in the foldable walls. Possibly, the top is resilient to allow depression of the pocket to force release of gas through the gas release vents.

Potentially, the filter vent includes a scenting agent.

Potentially, the filter vent is directly aligned with the stoma opening.

Further in accordance with certain aspects of the present invention there is provided a stoma closure arrangement comprising a patch with an outlet aperture for release of faeces, the outlet aperture closable with a cap and the cap having vents to release gas.

Generally, the cap incorporates a filter for gas. Furthermore, the filter incorporates a deodorising agent.

Typically, the cap is disposable once removed from closure of the outlet aperture. Possibly, the cap is formed from a cardboard or compressed paper material.

Alternatively, the cap is washable for re-use in order to close the outlet aperture. Consequently, the cap may be formed from a plastics material.

Advantageously, the outlet aperture has a screw thread to secure the cap. Possibly, the screw thread allows a collection bag to be secured to the outlet aperture. Further advantageously, the screw thread allows an evacuation pump to be secured to the outlet aperture. Alternatively, the outlet aperture incorporates a detent for interference engagement with the cap.

Typically, the cap is smoothly shaped for a limited depth profile. Possibly, the cap is arranged to be consistent with a dome cross-section across the arrangement.

Possibly, the outlet aperture has a concertina wall for expansion under faeces or gas pressure to provide an indication of a faeces evacuation requirement. Typically, the certain wall is compressible when expanded when expansion is due to gas pressure. Furthermore, the cap may be secured upon the outlet aperture to allow displacement of an association between the cap and the outlet aperture under faeces or gas pressure.

Possibly, the cap is adjustable to vary the size of the vents for release of gas or alternatively increase the number of vents available for release of gas.

Aspects of the present invention will now be described by way of example only with reference to the accompanying drawings in which:

FIG. 1 is a schematic side view of a bag arrangement in accordance with a first embodiment of certain aspects of the present invention;

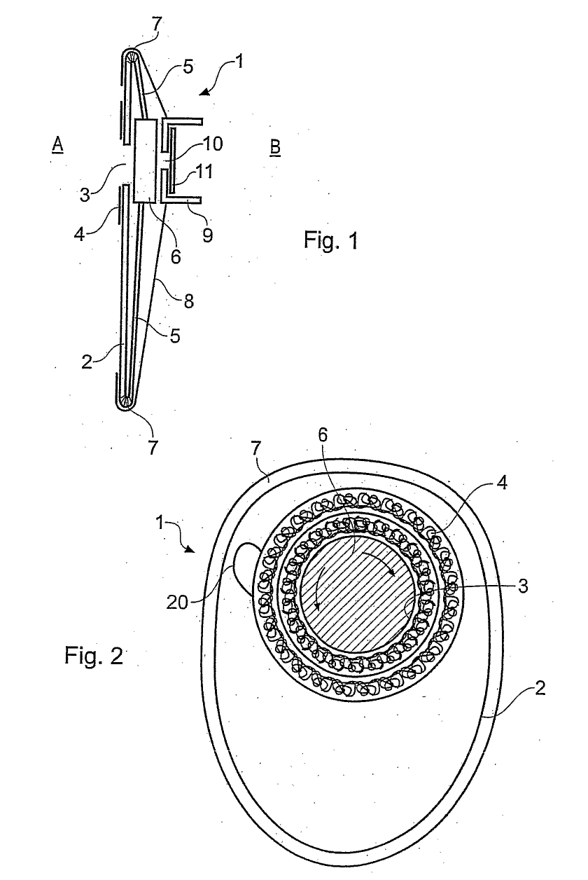

FIG. 2 is a schematic view of a stoma opening side of the bag depicted in FIG. 1;

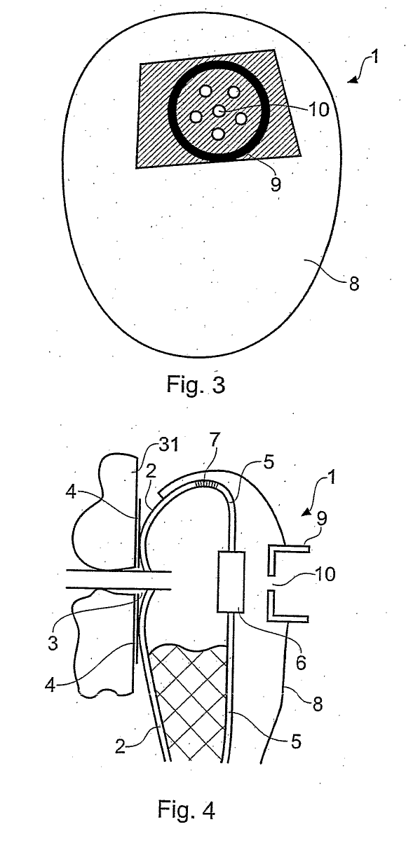

FIG. 3 is a schematic plan view of an adaptor side of the bag arrangement depicted in FIGS. 1 and 2;

FIG. 4 is a schematic cross section of a bag arrangement as depicted in FIGS. 1 to 3 in use;

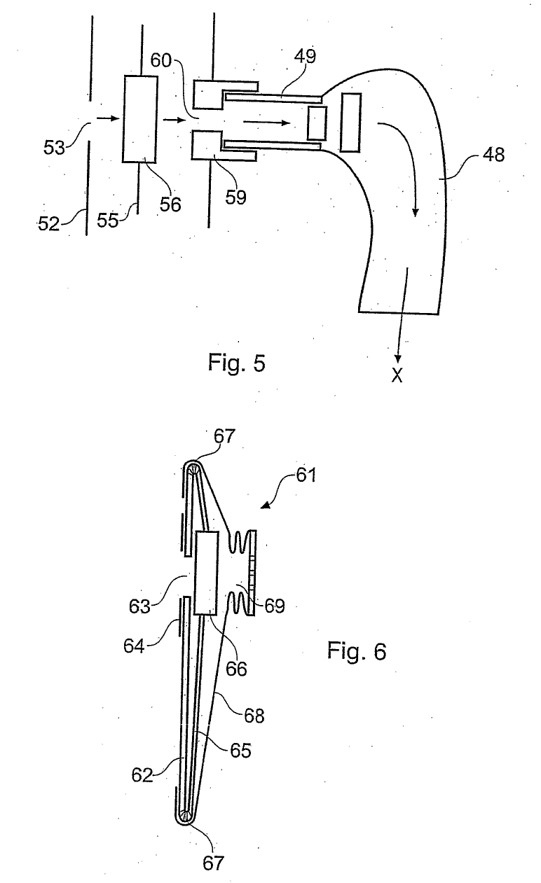

FIG. 5 is a schematic illustration of a method and process by which bowel movement training and control is facilitated;

FIG. 6 is a schematic side view of a bag arrangement in accordance with a second embodiment of certain aspects of the present invention;

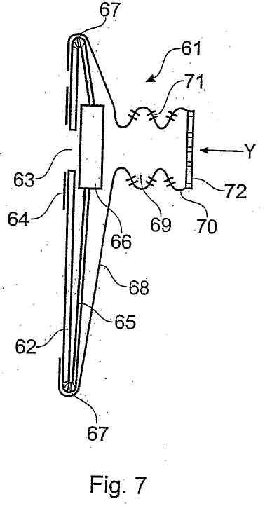

FIG. 7 is a side perspective view of the bag arrangement depicted in FIG. 6 in use;

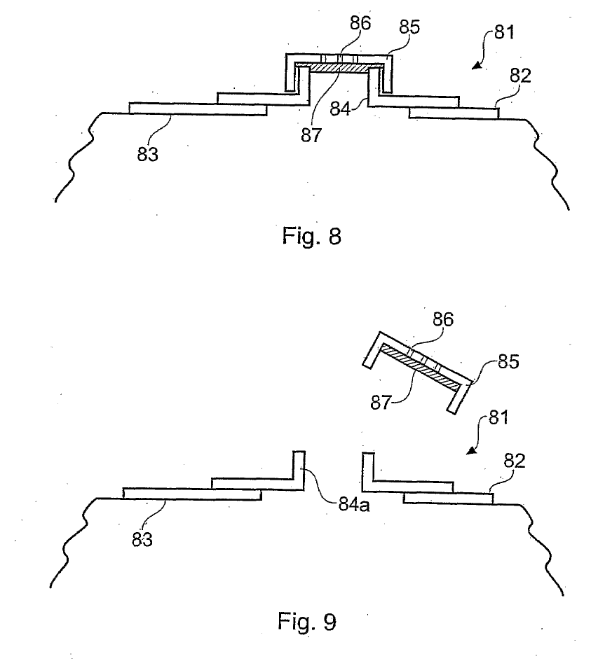

FIG. 8 is a schematic cross section of a stoma closure arrangement in accordance with aspects of the present invention; and,

FIG. 9 is a schematic cross-section of the arrangement depicted in FIG. 8 with a cap removed to allow evacuation of faeces

Convenient use of bags by association with a stoma exit after colostomy surgery is important in order to maintain patient dignity and to allow those patients to continue with as normal a lifestyle as possible. Such convenience involves allowing predictability with respect to bowel movements and when such bowel movements occur allowing convenient accommodation of such movements.

It will be understood that typically in a user gravity plays a significant part in stimulating bowel movements but such influence may be more limited once colostomy surgery has been performed. In such circumstances, regaining understanding and predictability with regard to bowel movement is important to an individual who has had colostomy surgery. Waste within an intestine will generally comprise agglomerations of waste digestion solids as well as gases and liquids. Gases can present problems as will be described later which must be accommodated but also can be utilised in order to stimulate bowel movements. It will be understood that the gases and potentially some liquids are more mobile than agglomerated solids. In such circumstances by stimulating gas movement and removal from the bowel wastes that this may cause some breakdown to co draw the solids, liquids and gas towards a stoma exit and through a stoma opening in the bag.

It will be understood as a result of colostomy surgery generally a stoma exit opening will be formed to extend through the abdomen of a patient. This stoma opening provides an outlet for wastes. Typically, the stoma exit is a plastic or other element surgically secured to the intestine and projecting in the order of 10 to 15 mm beyond the abdomen to provide a conduit through which wastes can be excised. Clearly, these wastes must be contained and generally a stoma bag is secured over the stoma to receive the wastes. Natural digestive reflexes and peristaltic motions may force the wastes into the bag. Nevertheless, particularly during the early stages of bowel movement training, it is difficult for a patient to achieve control of their bowel movements and therefore accidents and embarrassment can occur. One potential source of such irregularity is gas build up within the remainder intestine system introducing unpredictability with respect to bag expansion requirements and flow rates.

Certain aspects of the present invention provide a bag arrangement which can be associated with a method and process for bowel movement control. FIG. 1 provides a schematic side view of a bag arrangement 1 in accordance with certain aspects of the present invention. The arrangement 1 comprises a bag which is generally wet proof and therefore formed from a plastics or other material. This bag will receive the intestinal waste for subsequent disposal. The bag comprises a face sheet 2 in which a stoma opening 3 is provided to allow the arrangement 1 to be secured to a stoma exit. Normally, adhesive patches 4 are provided to ensure that the arrangement 1 is adhered to the abdomen of a user and so retain association between the opening 3 and a stoma (not shown). The bag is formed with a backer layer 5 which in accordance with certain aspects of the present invention includes a vent filter 6.

The arrangement 1 also includes a hood or cover 8 which at least extends over the backer layer 5 incorporating the vent filter 6. It will be appreciated that this hood 8 may also extend around the ends and be associated with the welds 7 in order to provide a cover for the bag comprising layers 2, 5 in order to provide user comfort and discretion. The hood 8 is normally formed from a fibrous material which may take the form of textile or a non woven sheet for user comfort and touch. Within the hood 8 an adaptor 9 is provided. As will be described later this adaptor 9 allows a gas evacuation pump to be secured to the arrangement 1 in order to draw gas through the filter 6 and a stoma opening 3 in order to stimulate and control bowel movements.

As depicted in FIG. 1 generally the stoma opening 3, the filter 6 and the adaptor 9 with an opening 10 are all advantageously aligned with each other to create a gas draw path through the opening 3, filter 6 and adaptor opening 10.

It will be understood that providing the arrangement in a relatively sterile state is important and so typically a patch 11 will be securable over the opening 10.

The vent filter 6 generally allows gas to be drawn through it but presents a barrier to solid wastes and typically expands to limit liquid transmission across the filter 6. It will be understood that the arrangement 1 will be regularly replaced and therefore generally the arrangement will be replaced before any substantial liquid has passed across the filter 6.

The filter 6 is generally formed from a fibrous material of a suitable sanitary grade such as absorbent materials. The filter is generally formed from an air laid paper or non woven fabric or wool. As the flow is generally one way it is will be understood that the filter 6 could also incorporate an absorbent powder to further limit the possibility of liquid transfer across the filter in use. Even more advantageously the filter 6 may incorporate scenting agents in order to apply a masking scent to any gaseous flow across the filter 6 and therefore avoid potentially embarrassing odours.

Normally the filter 6 will be integrally secured with the backer layer 5 to make sure that the barrier does not rupture in use.

As will be described later gentle natural gas drawing through the adaptor 9, filter 6 and opening 3 must be achieved. In such circumstances the filter 6 and/or the adaptor 9 may be arranged to ensure that overly aggressive gas extraction does not occur. For example, the patch 11 may be arranged to allow the number of holes in the adaptor 9 to be varied. Furthermore, as indicated above generally the hood or cover 8 is formed of a fibrous or textile material and therefore may allow for some air ingress to limit the suction capacity and therefore aggressiveness of the drawing gas flow through the filter 6.

FIG. 2 provides a schematic view from the direction of side A of the arrangement depicted in FIG. 1. Thus, as can be seen a stoma opening 3 provides an aperture to which a stoma exit can be secured. The filter 6 can be seen through the opening 3 and the patch 4 located around the opening 3 can be seen with its storage cover secured. This storage cover will be removed by pulling a tag 20 in order to expose the adhesive layer of the patch 4 to allow it to be secured to a user's abdomen. In such circumstances, it will be noted that the aperture 3 and the patch 4 will generally be located to one side of the arrangement 1 such that in use wastes can drop through the opening into a sump portion of the bag for collection.

FIG. 3 illustrates the arrangement 1 depicted in FIGS. 2 and 3 from a side B depicted in FIG. 1. Thus, the arrangement 1 has a hood or cover 8 in which the adapter 9 is secured, again to one side of the arrangement 1 for consistency with the filter 6 and opening 3 (not shown). The adapter 3 in such circumstances in use will be presented outwardly from a user's abdomen and allow a gas evacuation pump to be secured to the adapter in use.

As indicated above, the adapter can comprise a variety of small perforations or holes in a pepper-pot formation to allow gas effusion through the filter 6. Alternatively, as depicted in FIG. 1 a single central hole can be provided depending upon operational requirements.

As indicated above, generally the adapter 9 and, in particular, its opening 10, filter 6 and stoma opening 3, will be aligned. However, where desirable, these components may be misaligned within the arrangement in order to provide an indirect gas flow path which may facilitate regulation of gas flow to avoid overly aggressive effusion of natural gas.

FIG. 4 provides a schematic cross-section of the bag arrangement 1 depicted in FIGS. 1 to 3 in use. Thus, the arrangement is secured on a stoma exit 30 projecting from a user's abdomen 31. Waste 32 has been forced or drawn through the stoma 30 into the bag comprising layers 2, 5. The filter 6 in the back of layer 5 has prevented liquid and solid wastes 32 escaping from the bag created by the layers 2, 5. The adapter 9 remains associated with the hood or cover 8. In such circumstances, the aperture 10, if allowed to remain open, will allow any gas passing through the filter 6 to escape. Alternatively, as described previously, a patch 11 may be secured across the opening 10 to prevent release of gas. Alternatively, this patch may be rendered relatively porous to allow slow release of gas build-up between the hood or cover 8 and the back of layer 5. In any event, as described above, generally the hood or cover 8 will be formed from a fibrous material and, therefore, it will be understood that this material will be porous and allow slow release of gas passing through the filter 6 and dwelling in the space between the back of 5 and the hood or cover 8. In order to improve acceptability, the fibrous material from which the hood or cover 8 is formed or a patch 11 secured across the aperture 10 may incorporate scenting agents to mask the smell of the gas.

An aspect of the present invention relates to stimulating bowel movements and draining with regard such bowel movements. In such circumstances, the bag arrangement 1 as described above with regard to FIGS. 1 to 4 can be utilised. However, a base method in accordance with aspects of the present invention comprises applying a funnel-like member over a stoma exit in order to draw gaseous flow through the stoma exit from an attached remaining intestine. This stimulation will act to co-draw the solid/liquid wastes by effusion of gas from these wastes towards the gas evacuation mechanism. Typically this mechanism will comprise a simple impeller air or gas pump with a relatively gentle gas drawing rate. Such pumps have been utilised previously for drawing ear wax through horn elements from human ears.

It will be appreciated with a base funnel and associated gas evacuation pump no consideration is made with regard to inadvertent spluttering of waste into the pump which might create an unsightly mess. In such circumstances a bag arrangement 1 as described in FIGS. 1 to 4 will be advantageous in providing a process by which natural gas evacuation can occur through the stoma exit whilst avoiding a spluttering of wastes.

FIG. 5 provides a schematic illustration of a method and process by which bowel movements can be stimulated and individual training with regard to achieving periodicity with respect to bowel movements. As indicated above, natural gases within the intestine system are a variable factor which can create problems with respect to stoma bags. In such circumstances, by evacuating natural gases within the remaindered intestine and bowel allow stimulation of bowel movements and waste flow. In FIG. 5, a filter 56 is positioned as previously in a backer layer 55 of a bag formed with a layer 52 incorporating a stoma opening aperture. A hood or cover 58 includes an adapter 59 having an aperture 60 in order to draw air and natural gas in the direction of arrowhead X through the filter 56, aperture 60 and into a funnel 49 associated with a gas withdrawal pump 48. The pump 48 includes an impeller driven by an electric motor in order to draw air in the direction of the arrowheads X along with natural gases through the filter 56. As described previously, generally the hood or cover 58 will be fibrous and therefore allow some external air leakage into the flow path as well as drawing natural gas through the filter 56 such that there is a gentle evacuation of gas. Typically, the funnel 49 is secured upon a mounting for the pump 48 which will include a wire mesh or other relatively open screen to prevent any fibrous or debris which could become detached entering the pump 48.

In use, as described previously, a bag arrangement will be secured upon a stoma through the stoma opening 53 and any closure patch or otherwise removed across the adaptor 59 in order to expose the aperture 60. The funnel 49 will then be secured to the adaptor 59 and the pump 48 switched on. Operation of the pump will generally be for a pre-determined time period which is normally in the order of five minutes. This time period may vary dependent upon requirements so that during a timing period as indicated more stimulation of bowel movement 2 gaseous withdrawal will be provided whilst if simple removal of actual gas flatulence is required operation of the pump may only occur for a short period of time. In either event, as described previously, drawing the natural gas from the waste will cause typically co-movement and, therefore, stimulate bowel movement. In any event, as will be appreciated, trapped wind can cause pain and, therefore, removal in accordance with the method and process of the present invention will be advantageous.

Once the pre-determined time period for gas evacuation has been performed, it will be understood that the pump 48 will be detached and typically a closure path or flap located across the aperture 60. However, natural gas pressure may still allow transmission of some gas across the filter 56 and this will be gradually released through the aperture 60 and potentially the fibrous open-pore nature of the hood or cover 48. It will be understood that the pump 48 may have an integral funnel 49 or other means of coupling to the adapter 59 including a bespoke male/female coupling or simple attachment nozzle.

It will be understood that the natural gases evacuated through the filter 56 may have an undesirable smell and in such circumstances the filter 56 may incorporate a scenting agent which will be picked up by the gas flow across the filter 56 in order to create a more pleasant odour. Furthermore, the hood or cover 58 may also incorporate a scent which may be actively picked up by the gas or provide a simple background pleasant odour.

It will be understood that generally the gas evacuated in accordance with the above method and process may have a humidity or moisture content. In such circumstances this moisture will tend to be collected by the filter 56 in order to act as a barrier to prevent such moisture being released. The filter 56 will, therefore, progressively become increasingly wetted. In such circumstances the filter 56 may incorporate absorbent powders or other means including absorbent fibres in order to reduce the moisture content in the gas evacuated through the aperture 60 into the pump 48. These fibres may also have anti-microbial and disinfectant actions. It will also be understood in view of the moisture content variation in the filter 56 it may be possible to provide an identifier when the filter 56 has become saturated or indication as to when it will be advisable to stop gas evacuation. This indication may take the form of a colour change in the filter 56 or potentially other parts of the bag arrangement. This colour change could be achieved through an absorbent agent which changes its colour between dry and wet states.

As indicated above, once the pump is removed, the bag stays in place secured to the stoma through the aperture 53 and as indicated previously typically an adhesive patch secured to the user's abdomen. In such circumstances, the bag formed by the layers 52, 55 can act as previously as a receptacle for waste passed through the stoma and collected normally. The filter 56 particularly when wet will act as a barrier to moisture and solids movement through the bag and, therefore, ensure containment of the waste. As indicated above, the filter 56 may be scented to further reduce any noxious odours. The filter 56 is generally integrally formed with the layer 55 to limit any potential for rupture at the junction between the filter 56 and the layer 55 forming the receptacle bag for wastes. Once the bag is full, it is removed in the normal way and disposed of appropriately.

It will be understood that stoma waste collecting bags associated with stomas can be relatively intrusive and, therefore, during normal daytime may be unacceptable. Collection bags are typically used overnight or at other convenient times whilst less intrusive bags are used for daytime periods. These daytime bags will be of a smaller nature and typically will be designed to accept one to three hours' waste bowel movements. In such circumstances the bags have a lower capacity but it will be understood that the voluminous nature of gas effluxions can create problems. In such circumstances it has been known to provide a small sponge-type element within bags which is designed to actively absorb the natural gas emissions. Furthermore, it has been known previously to provide a filter and cover which incorporate pin-hole apertures to allow slow release of natural gas. In accordance with some aspects of the present invention, a bag arrangement for colostomy stomas is provided which can more readily accommodate gas emissions.

FIGS. 6 and 7 respectively show a schematic cross-section a bag arrangement 61 in its initial state in FIG. 6 and in use in FIG. 7.

The bag arrangement 61 comprises a contact layer 62 incorporating a stoma aperture 63 and adhesive flap 64 to allow the bag arrangement 61 to be secured to a user's stoma. A backer layer 65 incorporates a filter vent 66 which as illustrated is most conveniently directly aligned with the stoma aperture 63. A receptacle bag is created through edge-welding 67 so that wastes passing through the aperture 63 are collected in the bag between the layers 62, 65. The filter 66 allows gas to pass through into a capture area formed by a cover 68. Again this cover 68 will generally be secured about the periphery of the arrangement 61 so that gas is contained. It will be understood that the gas will preferentially pass through the filter 66 but in situations where there is limited solid/liquid waste expansion of the bag will principally be achieved by the gas. Release of the gas into the collection area between the back of the backer layer 65 and the cover 68 will again be retained until released.

In accordance with certain aspects of the present invention the cover 68 incorporates a concertina pocket 69 to act as the capture area for the natural gas passing through the filter 66. Having a concertina form, it will be understood that moulded sides or walls 70 unfold to increase the captive capacity of the pocket 69 to allow the natural gas to be collected. In accordance with certain aspects of the present invention, apertures or perforations are provided in the concertina pocket so that when the concertina pocket is opened an external pressure force in the direction of arrowhead Y can be applied in order to force natural gas out of the pocket 69.

In the above circumstances, if the bag arrangement 61 is full of liquid/solid waste then depression in the direction of arrowhead Y will not reduce the size of the arrangement 61 and a user will, therefore, be aware that it is necessary to change their bag 61. Alternatively, if depression in the direction of arrowhead Y results in a collapse of the arrangement 61, as the natural gas is forced through the apertures 71 it will be known that expansion was due to that natural gas and, therefore, the bag can still be used for a period of time.

In order to reduce odours, the filter 66 as well as the material from which the cover 70 is formed may incorporate a scenting agent.

Normally, the concertina fold walls 70 will be relatively flexible to enable ease of opening to increase the size of the pocket 69. A top 72 to the pocket 69 will generally be more robust to enable adequate depression pressure Y to be applied. Apertures can be provided in the top 72 but most conveniently the apertures will be in the side walls 70 so that the apertures open for normal aspirate release of gas as well as to allow unobstructed depression by depression force Y such as user's fingers for release of natural gas.

In use the arrangement 61 as previously will comprise placing the arrangement 61 on a stoma exit through the stoma aperture 64. Location of the arrangement 61 will be achieved through adhesive patches 64 secured to a user's abdomen. Normal gas and waste effusions will occur through the aperture 63 into the bag created by the layer 62, 65 and gas presented in this bag can pass through the filter vent 66 into the pocket 69. As indicated, this pocket will expand with gas with the apertures in the pocket through normal aspiration slowly releasing gas. If a user becomes aware of the bag 61 expanding significantly, conditions within the bag can be determined through an initial gentle depression in the direction of arrowhead Y. If the depression results in significant resistance this will indicate that water and solid wastes have been collected in the bag formed by the layers 62, 65. If depression receives little resistance then it will be understood that the bag and, in particular, the pocket 69 are filled with gas and further slightly more forceful depression will release that gas through the apertures 71.

As indicated above, generally the natural gas will be released from the pocket 69 through apertures 71 formed in the walls 70 as well as the top 72 but it will also be understood that the material from which the layer 68 is formed may be a textile or other fibrous material and, therefore, pores within this material may slowly release natural gas. The material may be scented. It will be appreciated that expansion of the pocket 69 will occur when the rate of gas transmission through the filter 66 exceeds the normal aspirate natural gas rate released through the pores in the material of the cover 68 as well as any normal opening or apertures.

As indicated above it is not always convenient to wear a colostomy bag during the daytime in view of its potentially bulky nature and therefore detraction from a user's body profile under clothing. In such circumstances, blanker or stoma closure arrangements are provided in the form of patches which extend across the stoma but do not incorporate a bag. In such circumstances these blanker or stoma closure arrangements are simply a patch with adhesive on one side to allow the patch to be secured to a user's skin surface and generally have means to seal around a stoma end to close it as well as to provide an environmental barrier. Clearly, bodily functions will still continue and therefore faece waste as well as digestion gases will still pass along to the stoma exit. In such circumstances these blanker or stoma closure arrangements will simply be removed and the faeces deposited in a lavatory or otherwise. Previously, once a blanker or stoma closure had been used it was disposed of.

The above approach to blanker or stoma closure arrangements necessitates repeated removal of the arrangement which can cause irritation and soreness around the stoma exit site and as there will be frequent situations where pain and pressure is simply due to digestion gases, a number of not easily recyclable or disposed of patches will be removed when not necessary.

A stoma closure arrangement 81 in accordance with aspects of the present invention is depicted in FIGS. 8 and 9 as a schematic cross-section. This arrangement 81 comprises a patch 82 which will be secured to a user's skin through an adhesive surface 83 about a stoma site. An outlet aperture 84 is formed in an appropriate structure secured to the patch 82. This outlet aperture 84 will allow release of faeces when a cap 85 is removed. In such circumstances the outlet aperture 84 is closable by the cap 85 to create a barrier across a stoma exit blanked off by the arrangement 81.

The cap 85 in accordance with aspects of the present invention incorporates vents 86 and a filter 87. In such circumstances with the cap 85 closing the aperture 84 it will be understood that gas will be released through the vents 86. The filter 87 is preferably provided in order to filter the indigestion gases to avoid release of waste particulars and moisture. The filter 87 may also incorporate a deodorising agent to render the indigestion gases more pleasant. It will be understood that the filter 87 may be removed if problems with respect to waste particulates, odour and moisture release as well as odour are not a concern, and the vents 86 will provide an adequate environmental barrier.

The cap 85 is secured to the outlet 84 generally through an upstanding portion. This upstanding portion 84a will incorporate a fastening mechanism generally upon its outer surface. This fastening mechanism will typically be a screw thread with a reciprocal screw thread in the cap 85 to allow the cap 85 to be secured across the aperture 84. Alternatively, the cap 85 may be an interference fit upon the upstanding portions 84 with an appropriate interference and detent engagement to prevent inadvertent release of the cap 85.

Where in particular a screw thread is provided on the upstanding portions 84 it will be understood that this fastening mechanism will be utilised to allow an evacuation pump similar to those described above to be secured across the aperture 84 in order to initiate through evacuation digestive motions. Additionally, the fastening mechanism may allow a bag to be secured over the aperture 84 for emergency use when access to a lavatory is not available.

As indicated above, a particular problem with previous blanker stoma closure arrangements was that these arrangements were formed from plastics materials which were less than convenient to dispose of. In such circumstances caps 85 in accordance with the present invention will generally be disposable at least to enable such caps to be flushed down a lavatory with faeces and other wastes evacuated through the aperture 84. Such disposability will typically be achieved through forming the caps 85 from cardboard or compressed paper. In such circumstances the problem with respect to placing such caps in dustbins for normal disposal are avoided. Alternatively, the caps 85 may be formed such that they are re-useable through a washing and sterilisation process if necessary. In such circumstances the caps may be formed from a plastics material or metal so that once removed the cap can be washed and replaced as required.

As indicated above, a principal advantage of blanker stoma closure arrangements is that they are less bulky than colostomy bags and so more acceptable for everyday use. In order to provide consistency with this advantage the caps in accordance with the present invention will generally be shaped for a limited depth profile. In such circumstances the arrangements in accordance with aspects of the present invention will be less conspicuous to a user. The caps will be rendered as flat as possible to, as indicated, limit the depth profile above a user's skin surface adjacent a stoma exit site. It will also be understood that the caps may be shaped for consistency with a domed cross section across the arrangement such that the smooth aspect of such a dome cross-section is less conspicuous.

A clear difficulty with regard to colostomy and stoma provision is that a user may not be able to distinguish between a bowel movement and indigestion gas build up. The present arrangement allows release of such indigestion gases through the vents but clearly, particularly where a filter is used, the rate of release of gas may be limited. In such circumstances, and as described previously, the upstanding portions of the aperture may be formed with a concertina or folded wall. In such circumstances, when the stoma closure arrangement is subjected to faeces or gas pressure on the under surface of the cap the concertina or folded wall will expand to provide an indication of such pressure. As indicated, this pressure may be due to solid faeces or natural gas build up. In order to determine whether the pressure is due to faeces typically the concertina wall will be compressible when expanded due to gas pressure whilst resistance to such compression will be presented when there is solid faeces acting against the cap. In such circumstances the concertina or folded wall will generally pop up to provide an indication to a user of the necessity to evacuate the stoma or release any built up gas through compression etc.

As indicated, gas release is generally through vents in an upper closure surface of a cap. The size and distribution of these vents will be dependent upon usual requirements. Thus, different vents numbers and distribution and vent consolidated cross sectional areas may be provided by different caps dependent upon user requirements. These caps may be differentiated by colour or otherwise to enable a user to select the right vent availability for expected use from previous experience or expected conditions. All these caps will be inter-changeable with the outlet aperture to enable the user to switch as required. It will also be understood that the number of vents available may be varied through providing an adhesive label appropriately segmented upon an upper surface of the cap 85 so that portions of these labels can be removed to expose more vents as required. Furthermore, a cap may be provided which is rotatable or otherwise movable upon the base closure cap such that apertures in the over cap and in the base cap overlap to varying extends in order to alter the size and distribution of vents available for digestion gas release. In the above circumstances typically the cap 85 or an over cap will incorporate outside knurling in order to facilitate rotation.

Alterations and modifications to the present invention will be appreciated by those skilled in the art. Thus, for example, more than one filter or aperture position may be provided with regard to gas evacuation and release. The apertures may be circular or oval dependent upon particular requirements and deodoriser and absorbent gases and powders may be provided within the bags and bag arrangements.

Whilst endeavouring in the foregoing specification to draw attention to those features of the invention believed to be of particular importance it should be understood that the Applicant claims protection in respect of any patentable feature or combination of features hereinbefore referred to and/or shown in the drawings whether or not particular emphasis has been placed thereon.

1-57. (canceled)

58. A bag arrangement for a colostomy stoma, the bag arrangement comprising a bag having a stoma opening and a vent filter, the arrangement including a hood at least secured over the vent filter, the hood including an adaptor to allow gaseous withdrawal through the vent filter.

59. An arrangement as claimed in claim 58 wherein the hood substantially surrounds the whole arrangement with an aperture through which the stoma opening projects.

60. An arrangement as claimed in claim 58 wherein the adaptor is sealable.

61. An arrangement as claimed in claim 58 wherein the filter is located directly opposite the stoma opening.

62. An arrangement as claimed in claim 58 wherein the filter is directly aligned with the adaptor.

63. An arrangement as claimed in claim 58 wherein the filter is impregnated with a scenting agent.

64. An arrangement as claimed in claim 58 wherein the filter is integrally secured to the bag.

65. An arrangement as claimed in claim 58 wherein the filter provides a moisture barrier when wet.

66. An arrangement as claimed in claim 58 wherein the filter and/or the adaptor are arranged to regulate rates of gaseous withdrawal in use.

67. A method of stimulating bowel movements in a colostomy patient, the method using a funnel associated with a gaseous withdrawal pump, the funnel positioned relative to a stoma and the pump operated to draw gas through this stoma for a desired period of time to remove gas by effusion from accumulated bowel waste in a bowel and such effusion stimulating by co withdrawal movements of the bowel waste itself.

68. A method as claimed in claim 67 wherein the desired period of time is sufficient to draw gas for bowel waste de-gasification.

69. A process for stimulation of bowel movements and control, the process comprising a bag arrangement for a colostomy stoma, the bag arrangement comprising a bag having a stoma opening and a vent filter, the arrangement including a hood at least secured over the vent filter, the hood including an adaptor to allow gaseous withdrawal through the vent filter and a method of stimulating bowel movements in a colostomy patient, the method using a funnel associated with a gaseous withdrawal pump, the funnel positioned relative to a stoma and the pump operated to draw gas through this stoma for a desired period of time to remove gas by effusion from accumulated bowel waste in a bowel and such effusion stimulating by co withdrawal movements of the bowel waste itself, the bag positioned with the stoma opening over the stoma and the withdrawal pump secured to the adaptor to draw gas through the filter from the stoma to stimulate movement of the bowel waste towards the bag.

70. A process as claimed in claim 69 wherein the process includes selective use of the withdrawal pump in order to train bowel movements and control.

71. A bag arrangement for a colostomy stoma, the bag arrangement comprising a bag with a stoma opening and a vent filter for gas, the arrangement having a cover with a concertina pocket to receive gas from the vent filter by expansion and having gas release elements.

72. An arrangement as claimed in claim 71 wherein the gas release elements comprise holes and the holes are round or oblong to vary aperture widths dependent upon expansion of the concertina pocket.

73. An arrangement as claimed in claim 71 wherein the gas release elements comprise diffusion through the material of the cover.

74. An arrangement as claimed in claim 71 wherein a top of the concertina pocket is resilient to allow depression of the pocket to force release of gas.

75. A stoma closure arrangement comprising a patch with an outlet aperture for release of faeces, the outlet aperture closable with a cap and the cap having vents to release gas.

76. An arrangement as claimed in claim 75 wherein the outlet aperture has a screw thread to secure the cap.

77. An arrangement as claimed in claim 76 wherein the screw thread allows a collection bag to be secured to the outlet aperture.

78. An arrangement as claimed in claim 76 wherein the screw thread allows an evacuation pump to be secured to the outlet aperture.

79. An arrangement as claimed in claim 71 wherein the outlet aperture incorporates a detent for interference engagement with the cap.

80. An arrangement as claimed in claim 71 wherein the outlet aperture has a concertina wall for expansion under faeces or gas pressure to provide an indication of a faeces evacuation requirement.

81. An arrangement as claimed in claim 80 wherein the cap may be secured upon the outlet aperture to allow displacement of an association between the cap and the outlet aperture under faeces or gas pressure.

82. An arrangement as claimed in claim 71 wherein the cap is adjustable to vary the size of the vents for release of gas or alternatively increase the number of vents available for release of gas.
